(19) United States
(12) Patent Application Publication
(10) Pub. No.: US 2022/0378789 A1
(43) Pub. Date: Dec. 1, 2022

(54) METHODS OF ADMINISTERING ELAGOLIX IN ASSOCIATION WITH ARTIFICIAL REPRODUCTIVE TECHNOLOGIES

(71) Applicant: John Anthony Schnorr, Awendaw, SC (US)

(72) Inventor: John Anthony Schnorr, Awendaw, SC (US)

(21) Appl. No.: 17/331,505

(22) Filed: May 26, 2021

(51) Int. Cl. A61K 31/513; A61K 38/24; A61K 9/00

(52) U.S. Cl. CPC A61K 31/513; A61K 38/24; A61K 9/0019

(57) ABSTRACT

A method of administering elagolix to a patient in association with an artificial reproductive technology (ART) protocol involves orally administering 150 to 400 mg elagolix per day. In accordance with certain embodiments, the method further involves the co-administration of exogenous gonadotropins (rFSH or HMG) with a subsequent ovulatory trigger. In an embodiment, the patient is administered 150 to 200 mg Elagolix PO daily or twice per day. In an embodiment, the patient is administered 150 to 200 mg Elagolix PO per day at a dosing duration of a minimum of one day and a maximum of six days. In an embodiment, the ART protocol involves administering 150 to 200 mg Elagolix PO per day based upon a patient's transvaginal ultrasound and hormone levels.

### FIELD

The present disclosure pertains to the administration of GnRH receptor antagonists in a patient in association with artificial reproductive technologies.

### BACKGROUND

Controlled ovarian hyperstimulation (COH) during in vitro fertilization (IVF) was one of the major advances of assisted reproductive technology (ART) during the second half of the 20th century. COH was developed to stimulate a larger than normal number of ovarian follicles, with the end result being retrieval of multiple fertilizable oocytes. There are multiple different exogenous gonadotropin regimens available tailored to individual populations: recombinant FSH (rFSH) and human menopausal gonadotropins (HMG), for example, to first stimulate multiple follicle recruitment. During COH, it is necessary to suppress the luteinizing hormone (LH) surge which may occur prematurely before the leading follicle reaches the optimum diameter for triggering ovulation and thus completing multiple oocyte retrieval. Suppression of the LH surge is accomplished with gonadotropin releasing hormone (GnRH) analogues, either agonists or antagonists, in several possible protocols. Traditionally, GnRH agonists have played an important role in reducing premature LH surges by reversibly blocking pituitary gonadotropin secretion. Agonists show a high rate of efficacy but require several weeks of desensitization, as the initial administration will cause a hormone flare with receptor activation. Additionally, agonist protocols increase overall cost due to an increased requirement for gonadotropin injections. GnRH antagonists were thus developed and FDA-approved to limit treatment to those days when a premature LH surge is likely to occur, as they cause immediate, reversible and dose-related inhibition of gonadotropin release, by competitive blockade of the GnRH receptors in the pituitary gland. Ganirelix and cetrorelix are two common, peptide, GnRH antagonists used in ovulation suppression during ovarian hyperstimulation. All GnRH analogues approved for use in assisted reproduction are peptides requiring subcutaneous (SQ) injections, in addition to the injectable rFSH used for follicle recruitment, making COH an injection-heavy endeavor for patients and donors. The recent release of an oral, non-peptide, GnRH antagonist presents an opportunity to decrease the number of injections, as well as lower the cost, involved in oocyte donation for in vitro fertilization.

4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid sodium salt (“Elagolix”) is an orally bioavailable, non-peptide gonadotropin-releasing hormone (GnRH) receptor antagonist FDA-approved for management of moderate to severe endometriosis pain. It binds competitively to and blocks the GnRH receptor in the anterior pituitary gland inhibiting endogenous GnRH stimulation, which rapidly suppresses luteinizing hormone (LH) and follicle stimulating hormone (FSH) secretion. This reduces estradiol and progesterone production in a dose-dependent manner. Elagolix is FDA-approved for management of pelvic pain in endometriosis, a condition in which estrogen is thought to promote the inflammatory implantation of endometrial-like tissue outside the uterus, causing dysmenorrhea, dyspareunia, infertility, and other less common symptoms.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

Certain aspects of the present disclosure provide for a method of suppressing ovulation in a patient, where the method includes orally administering to the patient once or twice daily 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid as a sodium salt (elagolix), where the sodium salt is administered in an amount equivalent to 150 to 400 mg of the free acid, wherein the elagolix is orally administered to the patient once or twice daily for a duration of one to six days according to an artificial reproductive technology protocol; and discontinuing the administration of the elagolix to the patient prior to administering an ovulatory trigger according to the artificial reproductive therapy protocol. In an embodiment, the ovulatory trigger may include a gonadotropin-releasing hormone agonist trigger.

In embodiments, the present disclosure provides a method of suppressing ovulation in a patient where an artificial reproductive technology protocol comprises a controlled hyperstimulation protocol.

In an embodiment, a method of administering elagolix to a patient may include co-administering human chorionic gonadotropin to the patient. The human chorionic gonadotropin may be subcutaneously administered to the patient once daily. The human chorionic gonadotropin may include a dose of at least 20 international units.

In embodiments, the present disclosure provides a method of suppressing ovulation in a patient where the artificial reproductive technology protocol includes discontinuing the administration of the elagolix to the patient prior to administering the ovulatory trigger to the patient. In some embodiments, the ovulatory trigger may be administered 24 hours prior to administration of the elagolix to the patient.

In an embodiment, a method of administering elagolix to a patient may include an artificial reproductive technology protocol that includes administering an oral contraceptive to a patient prior to administration of the elagolix to the patient, according to an ART protocol. In an embodiment, an ART protocol includes administering an oral contraceptive to a patient for 10 to 14 days prior to administering the elagolix to the patient.

In embodiments, the present disclosure provides a method of suppressing ovulation in a patient where the artificial reproductive technology protocol includes administering one or more subcutaneous injections of exogenous gonadotropins including human recombinant follicle stimulating hormone and human menopausal gonadotropin prior to administering the elagolix to the patient. In an embodiment, the one or more subcutaneous injections of exogenous gonadotropins including human recombinant follicle stimulating hormone and human menopausal gonadotropin may be administered subsequent to an oral contraceptive regimen, in accordance with an ART protocol.

In embodiments, the present disclosure provides a method of suppressing ovulation in a patient where the artificial reproductive technology protocol includes beginning the oral administration of the elagolix to the patient once or twice daily in response to performing a transvaginal ultrasound on the patient, where a 14 mm lead follicle is noted on the transvaginal ultrasound.

In embodiments, the present disclosure provides a method of suppressing ovulation in a patient where the method includes orally administering a dose of elagolix to the patient once or twice daily, where the sodium salt is administered in an amount equivalent to a total daily dose of 150 mg to 400 mg of the free acid.

Certain aspects of the present disclosure provide for a method of suppressing ovulation in a patient, where the method includes orally administering to the patient once or twice daily 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid as a sodium salt (elagolix), wherein the sodium salt is administered in an amount equivalent to 150 to 200 mg of the free acid, wherein the elagolix is orally administered to the patient once or twice daily for a duration of one to six days according to an artificial reproductive technology protocol; co-administering to the patient exogenous gonadotropins and discontinuing the administration of the elagolix to the patient prior to administering an ovulatory trigger to the patient according to the artificial reproductive therapy protocol.

Certain aspects of the present disclosure provide for a method of suppressing ovulation in a patient, where the method includes orally administering to the patient once or twice daily 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid as a sodium salt (elagolix), wherein the sodium salt is administered in an amount equivalent to 150 to 200 mg of the free acid, wherein the elagolix is orally administered to the patient once or twice daily for a duration of one to six days according to an artificial reproductive technology protocol; co-administering exogenous gonadotropins and discontinuing the administration of the elagolix to the patient prior to administering an ovulatory trigger to the patient according to the artificial reproductive therapy protocol, wherein the artificial reproductive technology protocol comprises beginning the oral administration of the elagolix to the patient once or twice daily in response to performing a transvaginal ultrasound on the patient, wherein a 14 mm lead follicle is noted on the transvaginal ultrasound.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The skilled artisan will understand that the figures, described herein, are for illustration purposes only. The inventive methods of the present disclosure may be better understood from the following illustrative description with reference to the following figures in which:

FIG. 1 is a density plot of an outcome scale of mature and total egg counts for elagolix administered in association with ART versus a control group, in accordance with certain aspects of the present disclosure;

FIG. 2 is a log outcome scale of mature and total egg counts for elagolix administered in association with ART versus a control group, in accordance with certain aspects of the present disclosure;

FIG. 3 is a box plot of mature and total egg counts for elagolix administered in association with ART versus a control group, in accordance with certain aspects of the present disclosure;

FIG. 4 is a box plot of a log outcome scale of mature and total egg counts for elagolix administered in association with ART versus a control group, in accordance with certain aspects of the present disclosure;

FIG. 5 is a density plot of a proportion of mature eggs by treatment for elagolix administered in association with ART versus a control group, in accordance with certain aspects of the present disclosure;

FIG. 6 is a bar graph of dosage frequency by treatment for elagolix administered in association with ART versus a control group, in accordance with certain aspects of the present disclosure;

FIG. 7 is a bar graph of elagolix distribution administered in association with ART, in accordance with certain aspects of the present disclosure;

FIG. 8 is a box plot of cumulative gonadotropin distribution by treatment for elagolix administered in association with ART versus a control group, in accordance with certain aspects of the present disclosure;

FIG. 9 is a bar graph of total days of gonadotropin distribution for elagolix administered in association with ART, in accordance with certain aspects of the present disclosure;

FIG. 10 is a bar graph of cumulative cetrotide dose by treatment for a control group, in accordance with certain aspects of the present disclosure;

FIG. 11 is a density plot of hormone values by treatment over time for elagolix administered in association with ART versus a control group, in accordance with certain aspects of the present disclosure;

FIG. 12 is an outcome scale of estrogen levels over time including mean trajectories for elagolix administered in association with ART versus a control group, in accordance with certain aspects of the present disclosure;

FIG. 13 is an outcome scale of log estrogen levels over time including mean trajectories for elagolix administered in association with ART versus a control group, in accordance with certain aspects of the present disclosure;

FIG. 14 is a density plot of estrogen levels by treatment over time for elagolix administered in association with ART versus a control group, in accordance with certain aspects of the present disclosure;

FIG. 15 is a density plot of log estrogen levels by treatment over time for elagolix administered in association with ART versus a control group, in accordance with certain aspects of the present disclosure; and

FIG. 16 is a density plot of hormone values by treatment over time for elagolix administered in association with ART versus a control group, in accordance with certain aspects of the present disclosure.

### DETAILED DESCRIPTION

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Where possible, any terms expressed in the singular form herein are meant to also include the plural form and vice versa, unless explicitly stated otherwise. Also, as used herein, the term “a” and/or “an” shall mean “one or more,” even though the phrase “one or more” is also used herein. Furthermore, when it is said herein that something is “based on” something else, it may be based on one or more other things as well. In other words, unless expressly indicated otherwise, as used herein “based on” means “based at least in part on” or “based at least partially on.” Like numbers refer to like elements throughout. All definitions, as defined and used herein, should be understood to control over dictionary definitions, definitions in documents incorporated by reference, and/or ordinary meanings of the defined terms.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both of the endpoint limits, ranges excluding either or both of those included endpoints are also included in the scope of the invention.

As used herein, unless otherwise stated, definitions of the terms and illustrations of the techniques of the present disclosure may be found in any of several well-known references such as: F Olivennes, R Fanchin, P Bouchard, J Taieb, R Frydman, Triggering of ovulation by gonadotropin-releasing hormone (GnRH) agonist in patients pretreated with a GnRH antagonist, Fertility and Sterility, Vol 66, No. 1, July 1996. PMID: 8752628 DOI: 10.1016/s0015-0282(16)58404-0. Al-Inany H G, Youssef M A, Ayeleke R O, Brown J, Lam W S, Broekmans F J. Gonadotropin-releasing hormone antagonists for assisted reproductive technology. Cochrane Database Syst Rev. 2016; 4:CD001750. doi: 10.1002/14651858.CD001750.pub4. PubMed PMID: 27126581; Gordon K, Hodgen G D. GnRH analogues in ovarian stimulation. Ann N Y Acad Sci. 1991; 626:238-49. PubMed PMID: 2058957; Lyseng-Williamson K A. Elagolix in endometriosis-related pain: a profile of its use as approved in the USA. Drugs & Therapy Perspectives. 2019; 35(3):110-8. doi: 10.1007/s40267-019-00606-y; Lamb Y N. Elagolix: First Global Approval. Drugs. 2018; 78(14):1501-8. doi: 10.1007/s40265-018-0977-4. PubMed PMID: 30194661; PMCID: PMC6244606; Taylor H S, Giudice L C, Lessey B A, Abrao M S, Kotarski J, Archer D F, Diamond M P, Surrey E, Johnson N P, Watts N B, Gallagher J C, Simon J A, Carr B R, Dmowski W P, Leyland N, Rowan J P, Duan W R, Ng J, Schwefel B, Thomas J W, Jain R I, Chwalisz K. Treatment of Endometriosis-Associated Pain with Elagolix, an Oral GnRH Antagonist. N Engl J Med. 2017; 377(1):28-40. doi: 10.1056/NEJMoa1700089. PubMed PMID: 28525302; Giudice L C. Clinical practice. Endometriosis. N Engl J Med. 2010; 362(25):2389-98. doi: 10.1056/NEJMcp1000274. PubMed PMID: 20573927; PMCID: PMC3108065; Struthers R S, Nicholls A J, Grundy J, Chen T, Jimenez R, Yen S S, Bozigian H P. Suppression of gonadotropins and estradiol in premenopausal women by oral administration of the nonpeptide gonadotropin-releasing hormone antagonist elagolix. J Clin Endocrinol Metab. 2009; 94(2):545-51. doi: 10.1210/jc.2008-1695. PubMed PMID: 19033369; PMCID: PMC2646513; and, Ng J, Chwalisz K, Carter D C, Klein C E. Dose-Dependent Suppression of Gonadotropins and Ovarian Hormones by Elagolix in Healthy Premenopausal Women. J Clin Endocrinol Metab. 2017; 102(5):1683-91. doi: 10.1210/jc.2016-3845. PubMed PMID: 28323948.

As used herein, “exemplary” means serving as an example or illustration and does not necessarily denote ideal or best.

As used herein, the term “includes” means includes but is not limited to, and the term “including” means including but not limited to. The term “based on” means based at least in part on.

Unless otherwise specified, all documents referred to herein are incorporated by reference in their entirety.

Certain objects and advantages of the present disclosure provide for a method of administering an oral GnRH antagonist to decrease the number of injections involved in an assisted reproductive technology cycle. In certain embodiments, an assisted reproductive technology cycle may include oocyte donation for in vitro fertilization.

Certain objects and advantages of the present disclosure provide for a method of administering an oral GnRH antagonist to decrease the cost of medication involved in an assisted reproductive technology cycle.

Currently available GnRH antagonists are peptide formulations, a structure that necessitates frequent injections or implantation of long-acting depots. In contrast, Elagolix is a nonpeptide, small molecule GnRH antagonist that allows for oral bioavailability without sacrificing a high affinity for the GnRH receptor. Elagolix is rapidly absorbed after oral administration, with median time of maximum plasma concentration (Tmax) values ranging from 0.5-1 h. Maximum plasma concentration (Cmax) and total area under the curve (AUC) exposure are noted to increase in a dose dependent fashion. Elagolix concentrations decline with an estimated half-life (t½) of 4 to 6 hours.

Within an assisted reproductive technology cycle, final oocyte maturation and ovulation is required prior to oocyte retrieval with hormonal agents that have LH-like properties. There are two primary hormonal agents used to induce ovulation in an assisted reproductive technology cycle, GnRH agonists and human chorionic gonadotropin (HCG). The primary method for ovulation is the use of GnRH agonists which has the ability to produce final egg maturation and ovulation without the increased risk of ovarian hyperstimulation syndrome. The challenge of the use of a GnRH agonist for an ovulatory trigger in GnRH antagonist down regulated cycles is due to the possibility that the GnRH agonist is unable to dislodge the GnRH antagonist from the GnRH receptor thereby preventing the ability for the GnRH agonist to stimulate the GnRH receptor and result in ovulation. Frydman, et al, in 1996 determined in a prospective trial of patients with idiopathic infertility that patients receiving a subcutaneous, peptide GnRH antagonist could be administered a GnRH agonist successfully resulting in ovulation. Elagolix is an orally available, non-peptide GnRH antagonist with different pharmacokinetics than the currently used peptide GnRH antagonists that has not been studied in assisted reproductive technology cycles. As a result, the ability of a GnRH agonist to successfully stimulate a GnRH receptor blocked with an oral, non-peptide antagonist was previously unknown and unproven.

In accordance with certain aspects of the present disclosure, a method of administering an oral GnRH antagonist comprises an administration of elagolix per the oral route.

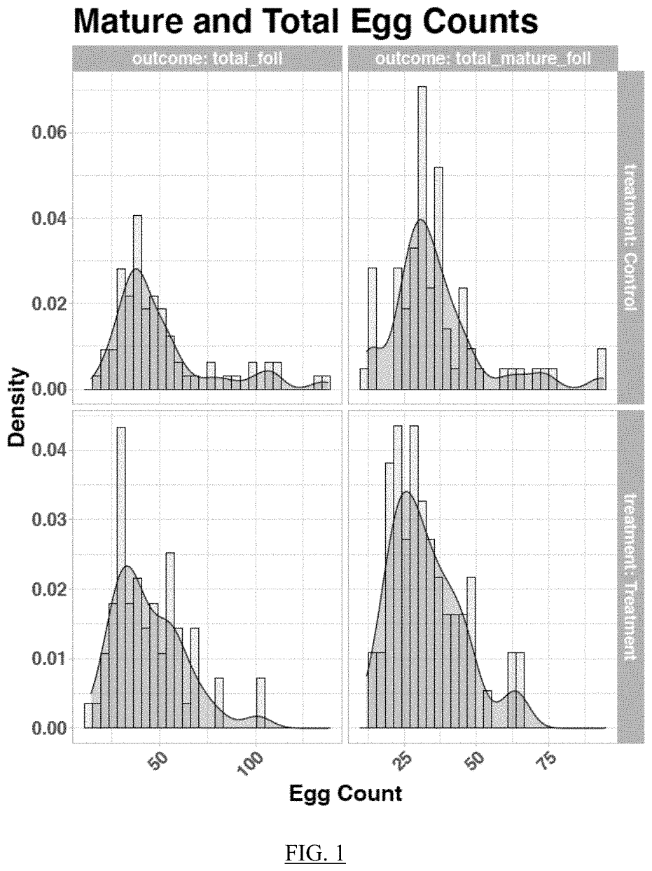

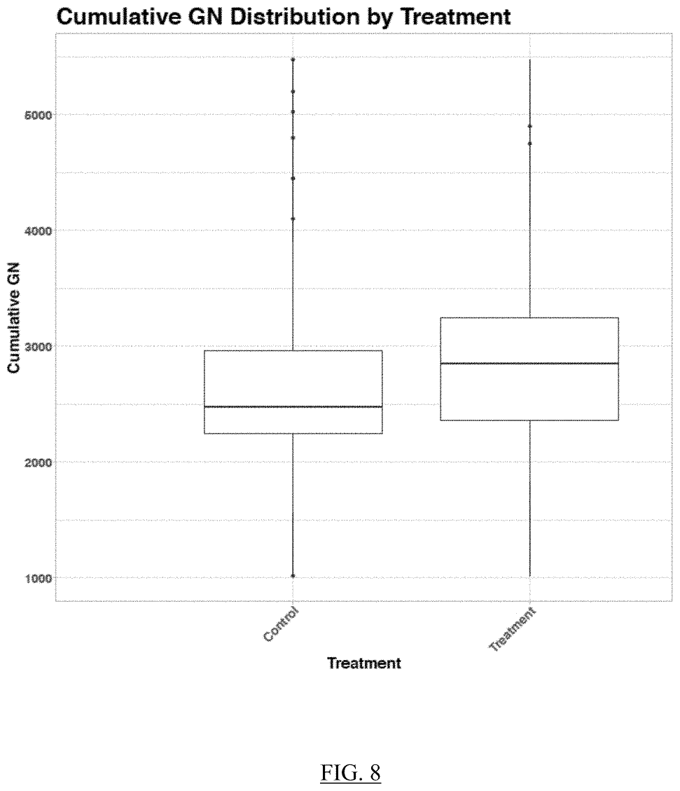

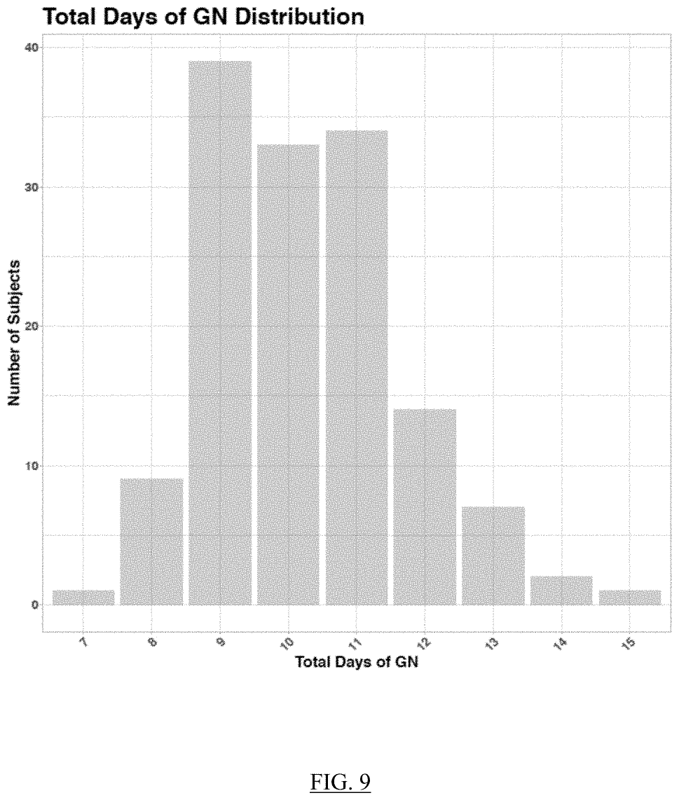

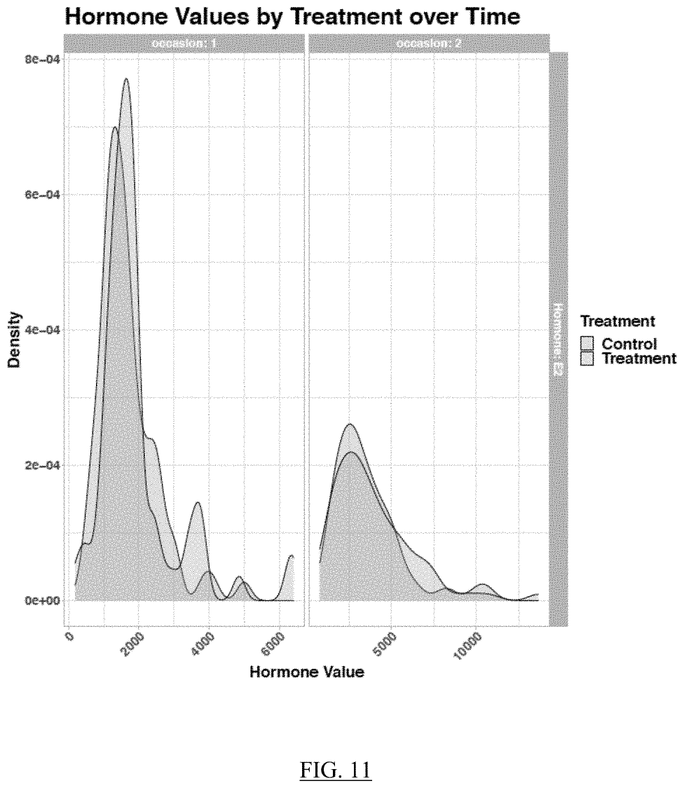

In accordance with certain embodiments, the present disclosure provides a method of administering elagolix to adequately suppress ovulation thereby decreasing the incidence of premature ovulation and simultaneously allowing for the successful administration of an ovulatory trigger during controlled ovarian hyperstimulation in ART. The primary outcome was the suppression of ovulation in an ART cycle as measured by the number of oocytes retrieved. Secondary outcomes of a method of the present disclosure include demonstration of an effective ovulatory trigger by measuring the number and proportion of mature oocytes (e.g., as shown in FIGS. 1-5), total amount of gonadotropins consumed (e.g., as shown in FIGS. 8-9), and maximum estradiol level (e.g., as shown in FIGS. 11-16).

In accordance with certain embodiments, the present disclosure provides a method of administering elagolix to a patient in association with an ART protocol. In an embodiment, the ART protocol may include administering daily SQ injections of rFSH or HMG with a dose based upon age, follicle stimulating hormone (FSH), antimullerian hormone (AMH) and antral follicular count to the patient after the onset of menstruation. In an embodiment, the ART protocol may include administering daily SQ injections of rFSH or HMG for four or more days. In an embodiment, the ART protocol may include obtaining estradiol levels and transvaginal ultrasounds during the administration of gonadotropins to monitor response. In an embodiment, the ART protocol may include adjusting a dosage of the rFSH or HMG according to the estradiol level for the patient. In an embodiment, the ART protocol may include obtaining an estradiol level and/or performing a transvaginal ultrasound for the patient at two-to-three-day intervals during administration of the daily SQ injections of gonadotropins. In an embodiment, the ART protocol may include adjusting a dosage of the gonadotropins according to an estradiol level and/or the transvaginal ultrasound.

In an embodiment, the method of administering elagolix to the patient in association with the ART protocol involves administering 150 to 200 mg Elagolix PO QD or BID. In an embodiment, the patient is administered 150 to 200 mg Elagolix PO QD or BID at a dosing interval frequency of 12-hours (e.g., 10:00 a.m. and 10:00 p.m.). In an embodiment, the patient is administered 150 to 400 mg Elagolix PO daily at a dosing duration of a minimum of one day and a maximum of six days. In an embodiment, the ART protocol involves administering 150 to 200 mg Elagolix PO QD or BID when a 14 mm lead follicle is noted on an ultrasound.

In an embodiment, a method of administering elagolix to the patient in association with an ART protocol includes determining a dosing interval frequency and/or a dosing interval duration according to one or more predetermined criteria for one or more of a follicular number, a follicular size, day of ovarian stimulation and an estradiol level for the patient. In an embodiment, a method of administering elagolix to the patient in association with an ART protocol includes modifying or adjusting (i.e., increasing or decreasing) a dosing interval frequency and/or a dosing interval duration according to one or more predetermined criteria for one or more of a follicular number, a follicular size and an estradiol level for the patient.

In an embodiment, a method of administering elagolix to the patient in association with an ART protocol involves discontinuing the administration of Elagolix prior to administration of an ovulatory trigger. In an embodiment, the ART protocol involves administration of an ovulatory trigger to induce ovulation in the patient. In an embodiment, the ART protocol involves retrieval of an egg subsequent to the administration of the ovulatory trigger.

In accordance with certain aspects of the present disclosure, i.e., a randomized, double blind, placebo-controlled, sequential dose escalation study, elagolix was well tolerated among healthy premenopausal women. No clinically significant adverse events occurred, and an acceptable safety profile was noted at doses up to 400 mg twice per day (BID). Oral bioavailability was rapid and serum levels of gonadotropins declined almost immediately, with LH levels of 22-35% baseline at 4 hours after administration of a single dose. Maximum inhibition of LH occurred in doses of 200 mg BID and above, with more sustained suppression at higher doses. This suppression was transient; at all dose levels, LH concentrations rebounded to about baseline levels or above within approximately 24 to 48 hours after the last dose. This data suggests that elagolix binds quickly and effectively to the GnRH receptor, but suppression was rapidly reversed after discontinuation. Suppression of estradiol also occurred within hours after the first dose, and similarly maximum suppression (to concentrations near 11.8 pg/mL) was achieved in all participants at elagolix doses of 200 mg BID and higher. In contrast to gonadotropins, estradiol suppression was more long lasting, with most groups remaining partially suppressed (42-65% of baseline) at 24 hours. Anovulatory concentrations were observed in all participants who received elagolix doses of 100 mg BID or higher and were maintained throughout the 7-day study with daily dosing.

Certain aspects of the present disclosure are described in greater detail in the non-limiting Example that follows.

### Example

An example of a method of the present disclosure includes a Western IRB approved (WIRB® Protocol #20191163) cohort study of 75 oocyte donors undergoing an assisted reproductive technology cycle receiving Elagolix 200 mg PO QHS for ovulation suppression compared to 75 historical donors given Ganirelix 250 mcg SQ QHS. Ovarian stimulation involved 10 days of oral contraceptive pretreatment. A recombinant FSH (Gonal F) starting dosage was based on donor age, AMH and BMI. 20 U HCG addback was given throughout the cycle with antagonists administered with a lead follicle of greater than 14 mm. The primary objective of this study was to determine if Elagolix adequately suppressed ovulation by measuring the premature ovulation rate during ovarian stimulation. Secondary outcomes are the number of mature oocytes at oocyte retrieval, the maximum estradiol level, the total amount of gonadotropins consumed, and the number of injections and patient cost.

### Study Medication:

The study medication was an orally bioavailable GnRH antagonist marketed by ABBVIE under the brand name ORILISSA® (Elagolix). The 200 mg dose was administered orally with once daily dosing. Contraindications included women with known osteoporosis or severe hepatic impairment (due to risk of bone loss), with concomitant use of strong organic anion transporting polypeptide (OATP) 1B1 inhibitors (e.g., cyclosporine and gemfibrozil), or strong Cytochrome P4503A (CYP3A) inhibitors (Clarithromycin, telithromycin, nefazodone, itraconazole, ketoconazole, atazanavir, darunavir, indinavir, lopinavir, nelfinavir, ritonavir, saquinavir, tipranavir).

### Data Analysis and Statistical Measures:

A study comprised 75 oocyte donors from whom data for 75 assisted reproductive technology cycles over the course of a two-year period was collected. Upon completion of data collection, a treatment group and an age and weight-matched control group were compared and assessed on the basis of demographic data, health history, ovarian reserve, and information regarding their ovarian stimulation cycles using Pearson χ2 and Student's t-tests. The primary outcome—premature ovulation rate—was evaluated using Chi squared tests. The secondary outcomes—number of mature oocytes, the maximum estradiol level, and the total amount of gonadotropins consumed—were compared using Student's t-tests.

### Study Participants:

All study participants were oocyte donors accepted into the oocyte donation program at Coastal Fertility Specialists in Mount Pleasant, S.C. Participants completed and passed the FDA, ASRM (American Society for Reproductive Medicine) and SART (Society for Assisted Reproductive Technology) approved evaluation of oocyte donors including history and physical examination, genetic screening, psychological screening and sexual transmitted disease (STD) screening. Participants were females 21 to 31 years of age without any history of STDs and with negative testing for HIV, Hepatitis B, C, Chlamydia and Gonorrhea. Participants did not have any contraindications to ovarian stimulation for oocyte donation. Exclusion criteria included contraindications to use of Elagolix, such as known osteoporosis or severe hepatic impairment (due to risk of bone loss), with concomitant use of strong organic anion transporting polypeptide (OATP) 1B1 inhibitors (e.g., cyclosporine and gemfibrozil), or strong Cytochrome P4503A (CYP3A) inhibitors (Clarithromycin, telithromycin, nefazodone, itraconazole, ketoconazole, atazanavir, darunavir, indinavir, lopinavir, nelfinavir, ritonavir, saquinavir, tipranavir).

Table 1 provides an overview of study enrollment including a number of subjects comprising 75 ovarian stimulations throughout a two-year period.

TABLE 1
STUDY ENROLLMENT
STUDY ENROLLMENT: Number of Subjects

| | Females | Males | Total |
|---|---|---|---|
| Ethnic Category | | | |
| Hispanic or Latino | 5 | 0 | 5 |
| Not Hispanic or Latino | 70 | 0 | 70 |
| Ethnic Category: Total of All Subjects* | 75 | 0 | 75 |
| Racial Categories | | | |
| American Indian/Alaska Native | 1 | 0 | 1 |
| Asian | 1 | 0 | 1 |
| Native Hawaiian or Other Pacific Islander | 1 | 0 | 1 |
| Black or African American | 10 | 0 | 10 |
| White | 62 | 0 | 62 |
| Racial Categories: Total of All Subjects* | 75 | 0 | 75 |

### Inclusion Criteria:

Women ages 21-32 years old; passed the FDA and American Society for Reproductive Medicine (ASRM) approved oocyte donor screening and medical consenting process; were approved by a physician for egg donation in an FDA-accredited oocyte donation program.

### Exclusion Criteria:

Absence of signed IRB approved consent to volunteer to participate in the study; known osteoporosis, severe hepatic impairment, or with concomitant use of strong organic anion transporting polypeptide (OATP) 1B1 inhibitors (e.g., cyclosporine and gemfibrozil); concomitant use of strong Cytochrome P4503A (CYP3A) inhibitors including Clarithromycin, telithromycin, nefazodone, itraconazole, ketoconazole, atazanavir, darunavir, indinavir, lopinavir, nelfinavir, ritonavir, saquinavir, tipranavir.

### Sources of Materials:

Research material obtained from living human subject's medical records included the following data: Age, FSH, AMH, antral follicle count; Estrogen levels; Number and size of follicles present on each ultrasound done; Number of eggs retrieved; Total amount of rFSH required.

Protocol for Oocyte Donation with Elagolix for Ovulation Suppression
1. Prospective donor accepted into FDA-accredited egg donation program after successful completion of screening and the signing of the medical consent forms.
2. Oocyte donor given opportunity to volunteer to participate in the study. Oocyte donor given an IRB approved informed consent document and HIPAA authorization for Oocyte Donation with Elagolix for Ovulation Suppression Protocol.
3. Oocyte donor begins oral contraceptives to lead into ovarian stimulation.
4. Oocyte donor discontinues oral contraceptives after 10 to 14 days of administration.
5. 3 days after discontinuing oral contraceptives, oocyte donor begins daily SQ injections of rFSH with the dose based upon age, follicle stimulating hormone (FSH), antimullerian hormone (AMH) and antral follicular count.
6. On day 4 of rFSH medications, the practitioner obtains an estradiol level from an oocyte donor. The rFSH dose will be adjusted as necessary.
7. At 2 to 3 days intervals of medications, the practitioner obtains an estradiol level and performs a transvaginal ultrasound for oocyte donor; an rFSH dose will be adjusted according to the results of the estradiol level and transvaginal ultrasound.
8. Once a 14 mm lead follicle is noted on the ultrasound, the practitioner administers 20 U per day of SQ hCG along with Elagolix 200 mg PO QD, taken at 10 pm. Elagolix is administered for a minimum of 1 day and a maximum of 6 days.
9. Administration of Elagolix 200 mg PO QD is discontinued 24 hours prior to the ovulatory, GnRH agonist trigger.
10. Ovarian stimulation is continued as outlined until oocyte maturity is reached based on follicular number, follicular size, cycle day and estradiol levels, at which time an ovulatory, GnRH agonist was used to induce ovulation and egg retrieval was performed per protocol. All mature eggs are either fertilized with the intended parent's sperm or cryopreserved for future used in egg donation.

### Results:

Oocytes were available in all retrievals as there were no instances of premature ovulation in either the Elagolix or Ganirelix groups. There were no statistically significant differences between groups in baseline demographics. Both groups had the same number of gonadotropins consumed, days of stimulation, number of oocytes and mature oocytes. The donors using Elagolix on average had 4.2 fewer injections per cycle with an average per cycle patient savings of $289.10. Table 2 provides a summary of a statistical comparison between a study group (Elagolix) and a control group (Ganirelix).

TABLE 2
Statistical Comparison between Study and Control Groups

| | Elagolix | Ganirelix | P-value |
|---|---|---|---|
| Mean Age: | 25.7 | 26.02 | 0.39 |
| Mean BMI: | 23.49 | 23.67 | 0.7 |
| Mean AMH: | 5.71 | 6.2 | 0.3 |
| Avg. Cum GN Dose: | 2855 | 2741 | 0.35 |
| Days of Stimulation: | 10.49 | 10.06 | 0.07 |
| Days of Antagonist: | 4.2 | 3.4 | 0.12 |
| Avg. Peak Estradiol: | 3959 | 3764 | 0.59 |
| Avg. Peak | 1.9 | 2.3 | 0.17 |
| Progesterone: | | | |
| Avg. Peak LH: | 0.94 | 1.3 | 0.69 |
| Avg. Oocytes: | 30.31 | 30.55 | 0.97 |
| Avg. Matures Oocytes: | 24.73 | 25.42 | 0.71 |

A density plot of an outcome scale of mature and total egg counts for the study group versus the control group is shown in FIG. 1.

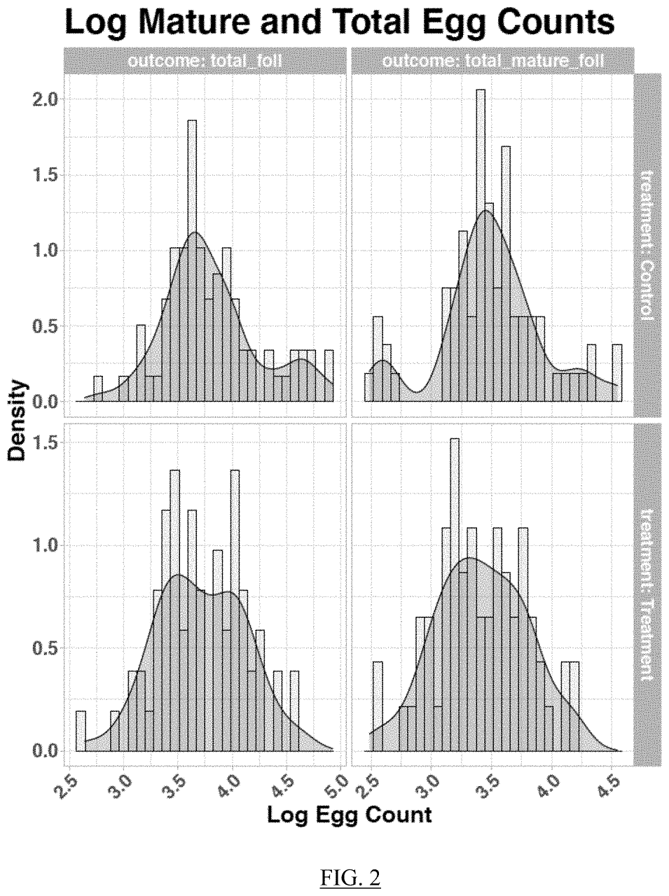

A log outcome scale of mature and total egg counts for the study group versus the control group is shown in FIG. 2.

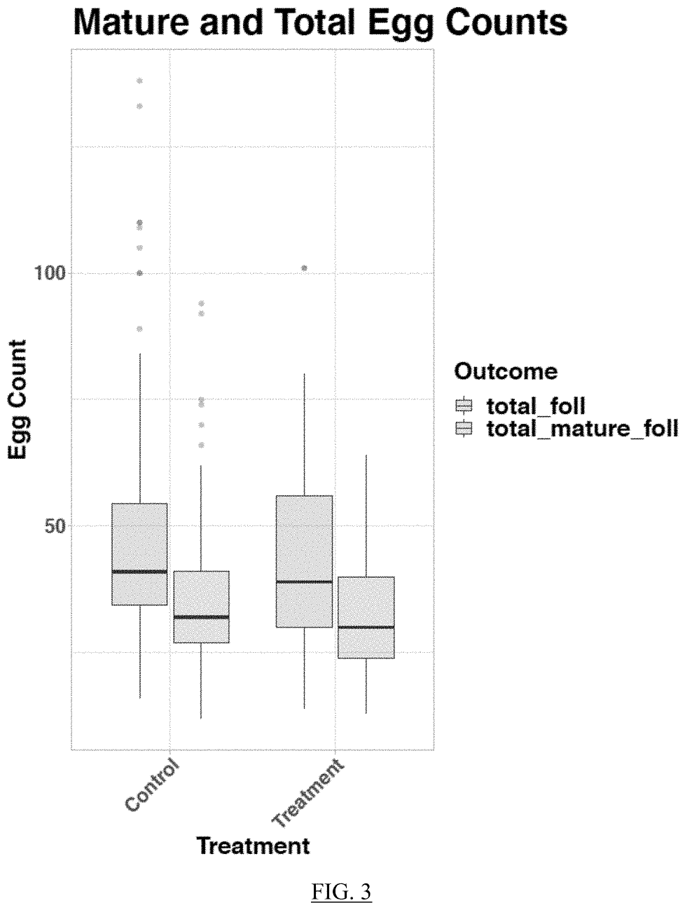

A box plot of mature and total egg counts for the study group versus the control group is shown in FIG. 3.

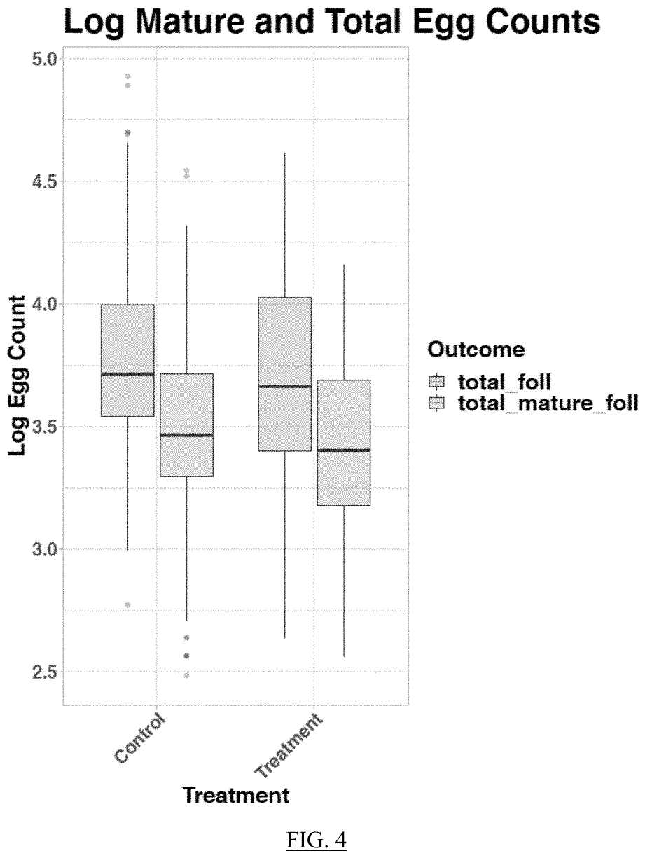

A box plot of a log outcome scale of mature and total egg counts for the study group versus the control group is shown in FIG. 4.

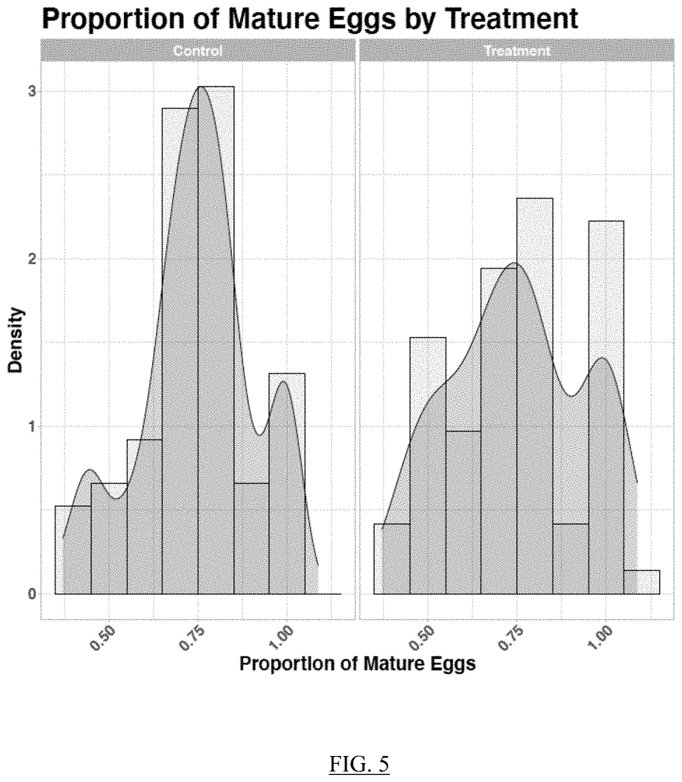

A density plot of a proportion of mature eggs by treatment for the study group versus the control group is shown in FIG. 5.

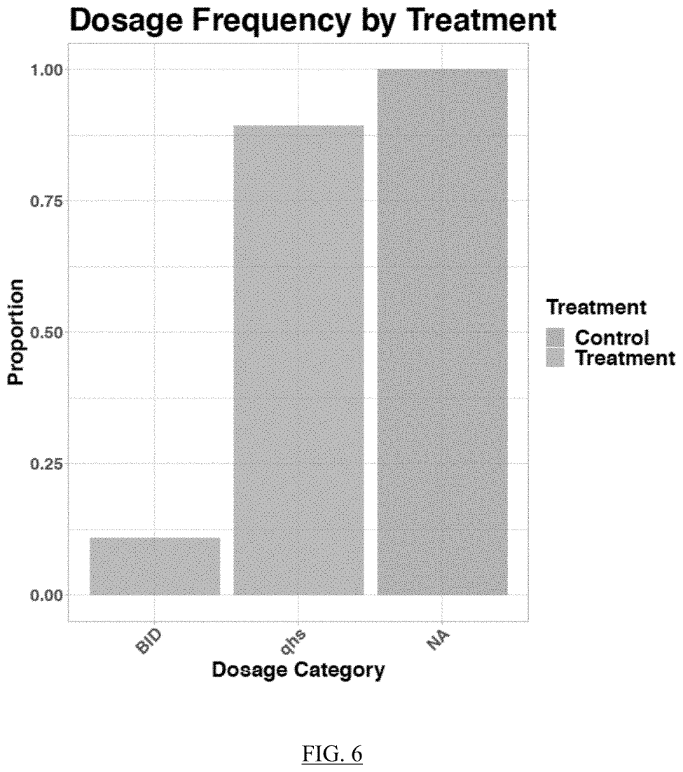

A bar graph of dosage frequency by treatment for the study group versus the control group is shown in FIG. 6.

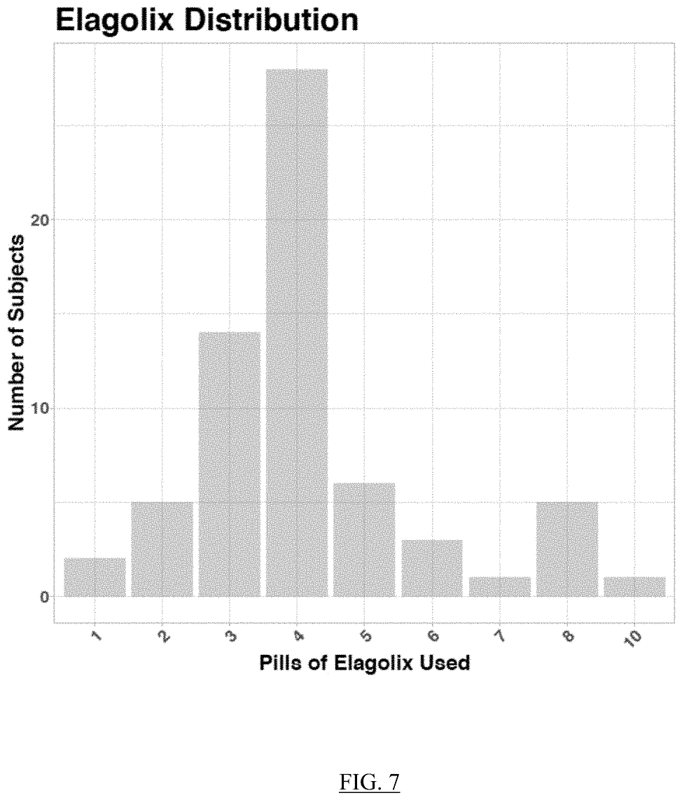

A bar graph of elagolix distribution for the study group is shown in FIG. 7.

A box plot of cumulative gonadotropin distribution by treatment for the study group versus the control group is shown in FIG. 8.

A bar graph of total days of gonadotropin distribution for the study group is shown in FIG. 9.

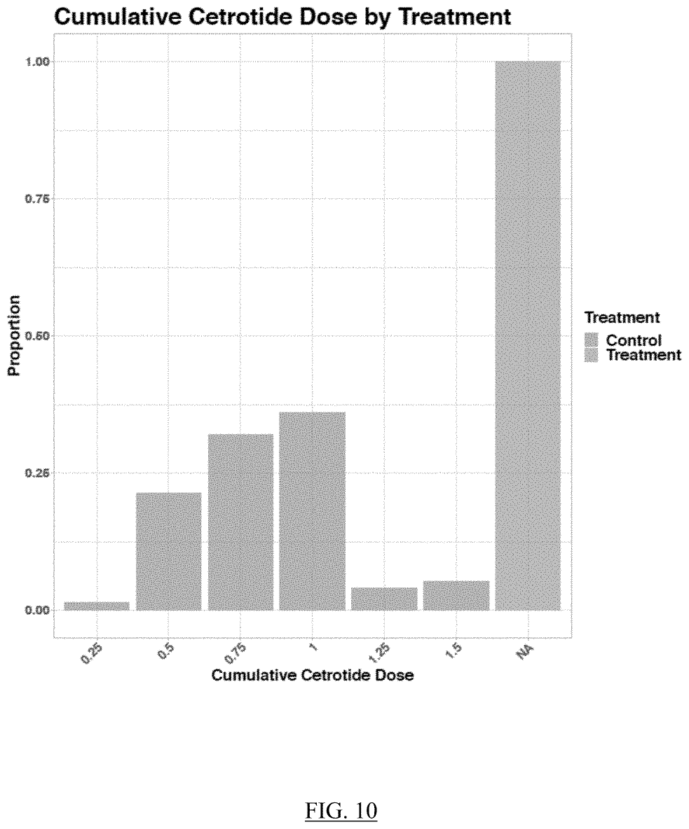

A bar graph of cumulative cetrotide dose by treatment for the control group is shown in FIG. 10.

A density plot of hormone values by treatment over time for the study group versus the control group is shown in FIG. 11.

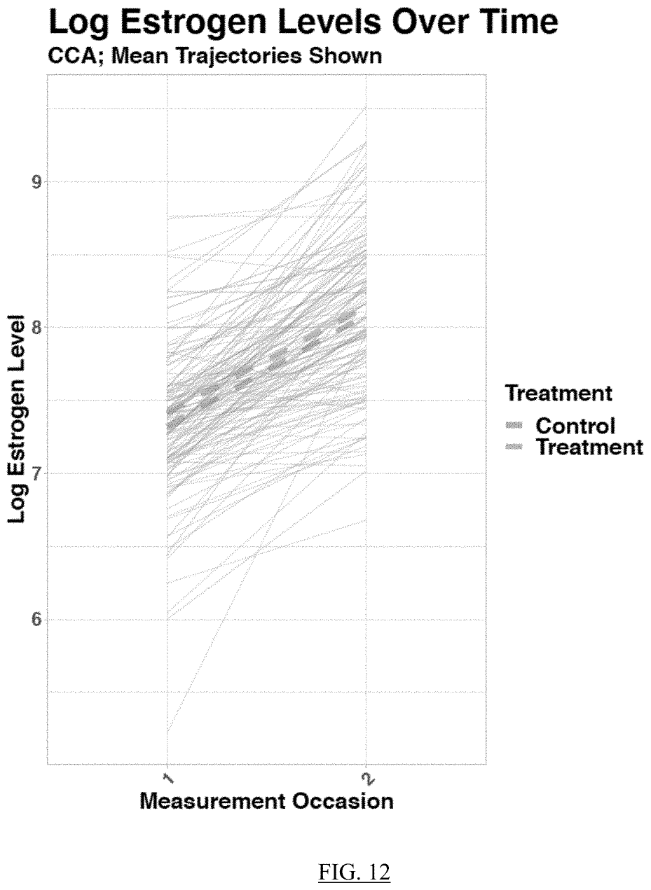

An outcome scale of estrogen levels over time including mean trajectories for the study group versus the control group is shown in FIG. 12.

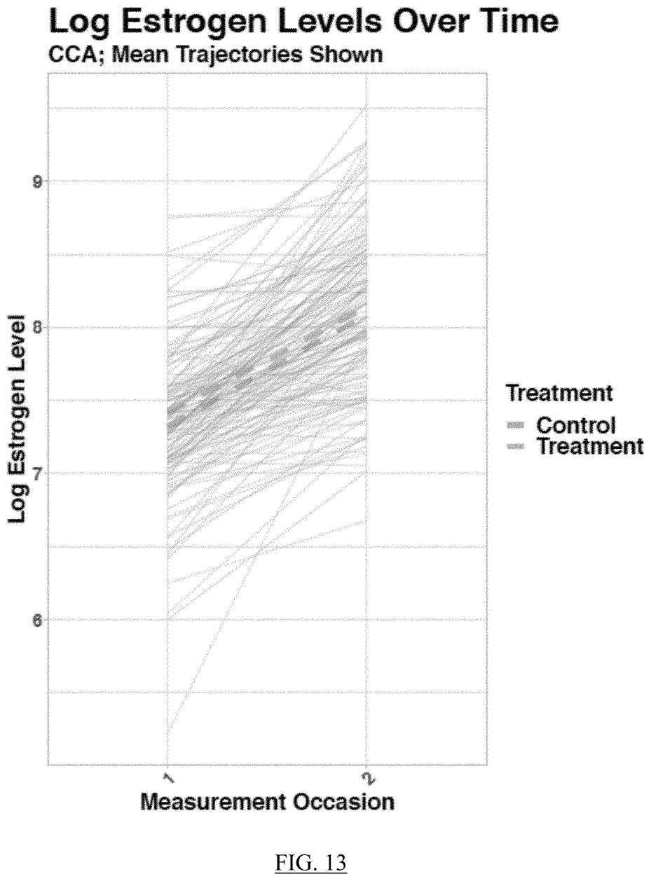

An outcome scale of log estrogen levels over time including mean trajectories for the study group versus the control group is shown in FIG. 13.

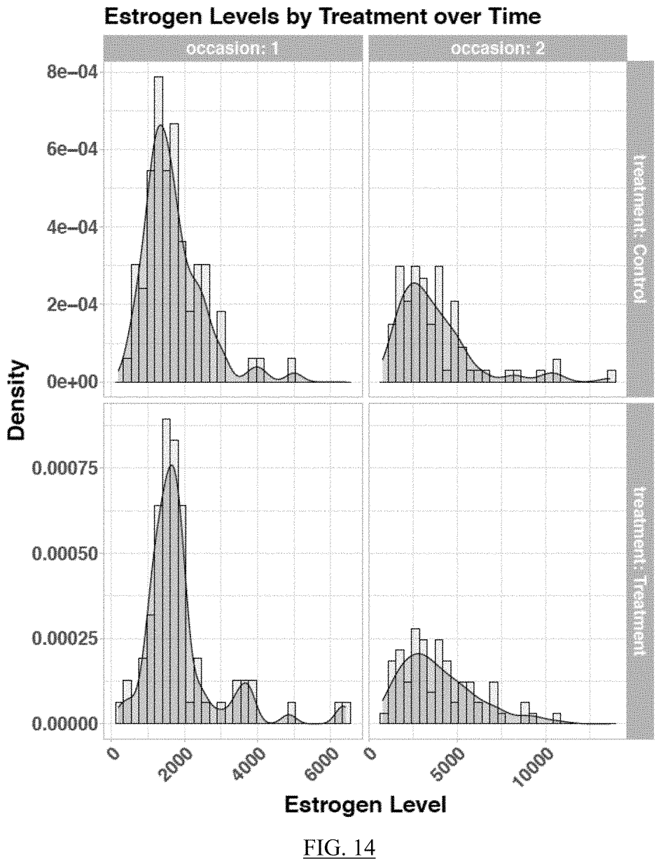

A density plot of estrogen levels by treatment over time for the study group versus the control group is shown in FIG. 14.

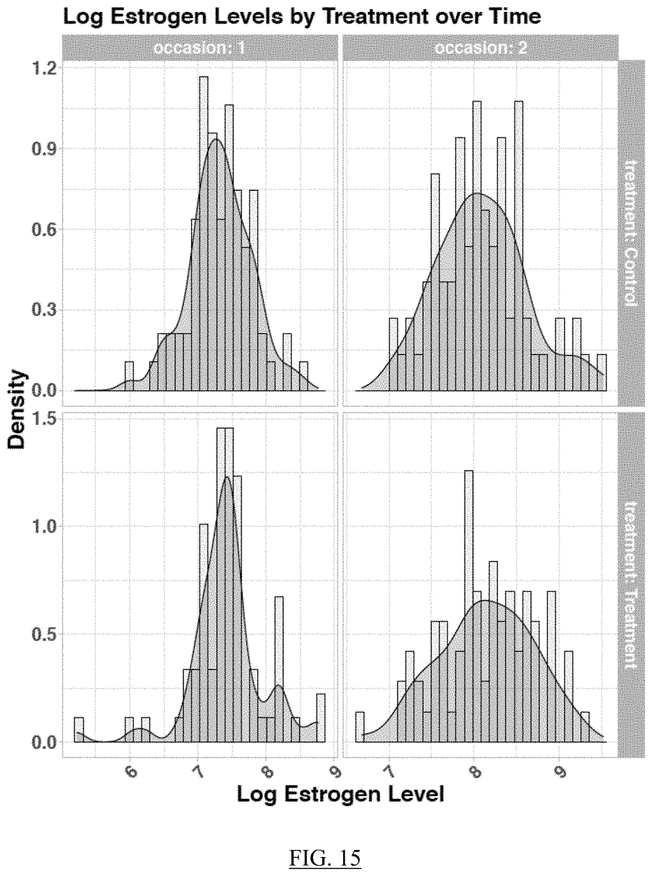

A density plot of log estrogen levels by treatment over time for the study group versus the control group is shown in FIG. 15.

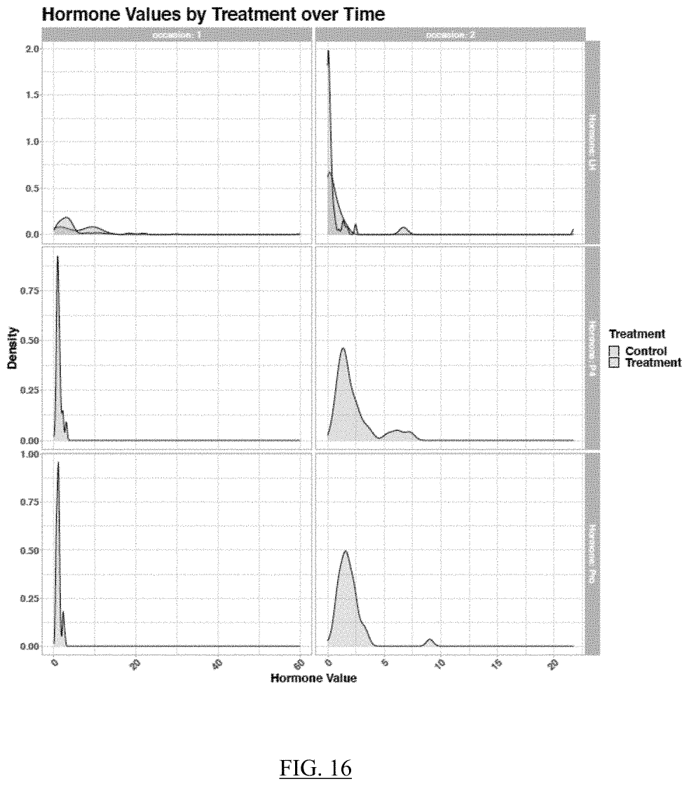

A density plot of hormone values by treatment over time for the study group versus the control group is shown in FIG. 16.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The present disclosure includes that contained in the appended claims as well as that of the foregoing description. Although this invention has been described in its exemplary forms with a certain degree of particularity, it is understood that the present disclosure has been made only by way of example and numerous changes in the details of construction and combination and arrangement of parts may be employed without departing from the spirit and scope of the invention. Therefore, it will be apparent to those skilled in the art that various modifications and variations can be made to the disclosed embodiments without departing from the scope or spirit of the invention. In view of the foregoing, it is intended that the invention covers modifications and variations of this disclosure within the scope of the following claims and their equivalents.

1. An artificial reproductive technology method for preparing a premenopausal female patient for oocyte retrieval in association with in vitro fertilization, the method comprising the steps of:
conducting a transvaginal ultrasound on the premenopausal female patient to identify a lead follicle for the premenopausal female patient;
subsequent to successfully identifying the lead follicle for the premenopausal female patient, orally administering to the premenopausal female patient once or twice daily 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid as a sodium salt (elagolix), wherein the sodium salt is administered in an amount equivalent to 150 to 200 mg of the free acid, wherein the elagolix is orally administered to the premenopausal female patient once or twice daily for a duration of one to six days;
discontinuing the administration of the elagolix to the premenopausal female patient after administering the elagolix to the premenopausal female patient for the duration of one to six days; and
administering an ovulatory trigger to the premenopausal female patient at least 24 hours after discontinuing the administration of the elagolix, wherein the ovulatory trigger comprises a gonadotropin releasing hormone agonist trigger.

2. The method of claim 1 wherein the elagolix is orally administered to the premenopausal female patient at a dosing interval frequency of 12 hours for the duration of one to six days.

3. The method of claim 1 wherein the lead follicle comprises a 14 mm follicle.

4. The method of claim 1 further comprising modifying a dosing interval duration of the elagolix according to at least one criteria for a follicular number, a follicular size and an estradiol level for the premenopausal female patient.

5. The method of claim 1 wherein the artificial reproductive technology protocol comprises administering one or more subcutaneous injections of exogenous gonadotropins to the patient prior to administering the elagolix to the patient.

6. The method of claim 5 wherein the exogenous gonadotropins comprise at least one of human recombinant follicle stimulating hormone and human menopausal gonadotropin.

7. The method of claim 1 wherein the artificial reproductive technology protocol comprises beginning the oral administration of the elagolix to the patient once or twice daily in response to a hormone level of the patient and performing a transvaginal ultrasound on the patient.

8. The method of claim 4 further comprising modifying a dosing interval frequency according to the at least one criteria for the follicular number, the follicular size and the estradiol level for the premenopausal female patient.

9. A method of suppressing ovulation in a patient, the method comprising:
orally administering to the patient once or twice daily 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid as a sodium salt (elagolix), wherein the sodium salt is administered in an amount equivalent to 150 mg to 200 mg of the free acid, wherein the elagolix is orally administered to the patient once or twice daily for a duration of one to six days according to an artificial reproductive technology protocol; and
discontinuing the administration of the elagolix to the patient prior to administering an ovulatory trigger to the patient according to the artificial reproductive technology protocol,
wherein administering the ovulatory trigger comprises administering a gonadotropin-releasing hormone agonist or human chorionic gonadotropin to the patient.

10. The method of claim 9 wherein the artificial reproductive technology protocol comprises a controlled hyperstimulation protocol.

11. The method of claim 9 wherein the artificial reproductive technology protocol comprises administering one or more subcutaneous injections of exogenous gonadotropins to the patient prior to administering the elagolix to the patient.

12. The method of claim 11 wherein the exogenous gonadotropins comprise at least one of human recombinant follicle stimulating hormone and human menopausal gonadotropin.

13. The method of claim 11 wherein the artificial reproductive technology protocol comprises administering the one or more subcutaneous injections of exogenous gonadotropins to the patient subsequent to menstruation and prior to administering the elagolix to the patient.

14. The method of claim 9 wherein the artificial reproductive technology protocol comprises beginning the oral administration of the elagolix to the patient once or twice daily in response to a hormone level of the patient and performing a transvaginal ultrasound on the patient.

15. The method of claim 9 wherein the sodium salt is administered in a range of an amount equivalent to 150 mg to 400 mg of the free acid.

16. A method of suppressing ovulation in a patient, the method comprising:
orally administering to the patient once or twice daily 4-((R)-2-[5-(2-fluoro-3-methoxy-phenyl)-3-(2-fluoro-6-trifluoromethyl-benzyl)-4-methyl-2,6-dioxo-3,6-dihydro-2H-pyrimidin-1-yl]-1-phenyl-ethylamino)-butyric acid as a sodium salt (elagolix), wherein the sodium salt is administered in an amount equivalent to 150 to 200 mg of the free acid, wherein the elagolix is orally administered to the patient once or twice daily for a duration of one to six days according to an artificial reproductive technology protocol; and
discontinuing the administration of the elagolix to the patient prior to administering an ovulatory trigger to the patient according to the artificial reproductive technology protocol,
wherein the artificial reproductive technology protocol comprises beginning the oral administration of the elagolix to the patient once or twice daily in response to a hormone level of the patient and performing a transvaginal ultrasound on the patient.

17. The method of claim 16 wherein orally administering the elagolix to the patient once or twice daily comprises administering a total daily dosage of the sodium salt in an amount equivalent to 150 to 400 mg of the free acid per day.

18. The method of claim 16 wherein the artificial reproductive technology protocol comprises administering one or more subcutaneous injections of exogenous gonadotropins to the patient subsequent to menstruation and prior to administering the elagolix to the patient.

19. The method of claim 16 wherein the artificial reproductive technology protocol comprises discontinuing the administration of the elagolix to the patient prior to administering the ovulatory trigger to the patient.

20. The method of claim 16 wherein the sodium salt is administered in a range of an amount equivalent to 150 mg to 400 mg of the free acid.
